(19) United States
(12) Patent Application Publication
(10) Pub. No.: US 2016/0249900 A1
(43) Pub. Date: Sep. 1, 2016

(54) ELASTIC TUBE, CONTROL DEVICE, AND MEDICAL EQUIPMENT

(71) Applicants: SHARP KABUSHIKI KAISHA, Osaka-shi, Osaka (JP); PUBLIC UNIVERSITY CORPORATION HIROSHIMA CITY UNIVERSITY, Hiroshima-shi, Hiroshima (JP)

(72) Inventors: Hitoshi AOKI, Osaka-shi (JP); Kazunori MORITA, Osaka-shi (JP); Kazuhiro TANIGUCHI, Hiroshima-shi (JP); Masazumi OKAJIMA, Hiroshima-shi (JP); Satoshi IWAKI, Hiroshima-shi (JP)

(21) Appl. No.: 15/030,928

(22) PCT Filed: Sep. 10, 2014
(86) PCT No.: PCT/JP2014/073943

§ 371 Date: Apr. 21, 2016

(30) Foreign Application Priority Data
Oct. 25, 2013 (JP) 2013-222672
Feb. 20, 2014 (JP) 2014-031079
May 30, 2014 (JP) 2014-113356

(51) Int. Cl. A61B 17/00; A61B 1/313

(52) U.S. Cl. CPC A61B 17/00234; A61B 1/3132; A61B 2018/2005

(57) ABSTRACT

An expansible elastic tube (1) in which gas is encapsulated, a medical instrument (an endoscopic camera 2) secured to the tip of the elastic tube (1), a non-expansive tube (3) that is connected to the other end of the elastic tube (1) in such a way as to communicate therewith, the non-expansive tube (3) in which gas is encapsulated, an inextensible body (4) having flexibility, the inextensible body (4) fixed to the elastic tube (1) in a longitudinal direction, a connecting tube (5) having the shape of a hollow circular cylinder and having flexibility and a non-expansive property, the connecting tube (5) connected to the non-expansive tube (3), and a control device (20) which performs variable control of the air pressure inside the elastic tube (1) are provided, and the air pressure is controlled by the control device (20), whereby the elastic tube (1) is made to expand and contract on the side opposite to the inextensible body (4) and the elastic tube (1) is curved at an arbitrary angle.

### TECHNICAL FIELD

The present invention relates to elastic tubes, control devices, and medical equipment. More particularly, the present invention relates to medical equipment as an endoscopic device which, for example, takes a photograph of an affected part at the time of surgery which is performed on a patient when medical procedures are performed, for example, an endoscopic device which is suitably used in single incision laparoscopic cholecystectomy. In addition, the present invention relates to medical equipment provided with a catheter, a laser scalpel, or an electric scalpel as a medical instrument, for example.

### BACKGROUND ART

Single incision laparoscopic cholecystectomy is a method of removing a gallbladder by opening, in a navel of a patient, one small hole which is about 15 mm and inserting forcipes (medical instruments) and an endoscopic camera through the hole. FIG. 12 is a front view depicting how existing surgery is performed. As depicted in FIG. 12, a surgeon (a camera assistant) 104 operates an endoscopic camera 103 and shows the state of the inside of a body of a patient 101 on a camera monitor 6. Another surgeon (an operator) 105 who is not the camera assistant 104 performs surgery by operating two forcipes 102, one held in a right hand and the other held in a left hand, while viewing the camera monitor 6. The endoscopic camera 103 comes around from a position above the forcipes 102 and takes images of an affected part of the patient 101. 8 denotes an operating table on which the patient 101 is placed.

Since single incision laparoscopic cholecystectomy leaves a scar only in a navel, single incision laparoscopic cholecystectomy has the following advantages.

(1) The scar is less noticeable, which offers cosmetic advantage.

(2) Single incision laparoscopic cholecystectomy causes less postoperative pain.

(3) A faster recovery time allows a patient to be discharged from the hospital after a shorter stay (2 or 3 days) than that in the existing surgery method, which makes it possible to curb medical costs.

(4) Even patients who have lost strength (elderly people) can have surgery safely because single incision laparoscopic cholecystectomy puts less physical burden on the body.

On the other hand, the following are problems of the existing single incision laparoscopic cholecystectomy.

(1) Contact between one person and another person and contact between surgery instruments occur outside the body, which hinders the movement of people and the instruments.

(2) Contact between the operator and the camera assistant.

(3) Contact between the forcipes and the endoscopic camera.

In order to solve the above-described problems, the tip part of the endoscopic device has to be formed to have a structure which allows the tip part to curve to take a photograph of a spot in an abdomen, the spot on which surgery is being performed. This curving structure is generally formed of a plurality of joints and a wire which operates the joints by pulling the joints. Moreover, as this curving structure, a structure which supplies fluid to the inside to make a film expand by the fluid pressure of the fluid and produces curving motion by tensile stress caused in the film is also known (Patent Literature 1). More specifically, a configuration which is a balloon actuator formed by combining two filmy bodies having different tensile stresses which are caused by expansion, the configuration in which, by forming one of the filmy bodies so as to have a concave shape, a plurality of spaces are partly formed between the two filmy bodies, is disclosed.

Moreover, in Patent Literature 2, for example, a configuration which produces curving movement by regulating the pressure inside a tubular body formed of a tubular elastic body and a reinforcing member which is less likely to be elastically deformed than the tubular elastic body is disclosed. In Patent Literature 3, a configuration provided with an air sac, a braid around the air sac, and a back column which is bent in an arching line and placed between the air sac and the braid is disclosed.

### CITATION LIST

### Patent Literature

PTL 1: Japanese Unexamined Patent Application Publication No. 2006-204612 (published on Aug. 10, 2006)

PTL 2: Japanese Unexamined Patent Application Publication No. 2-113104 (published on Apr. 25, 1990)

PTL 3: Japanese Unexamined Patent Application Publication No. 6-201958 (published on Jul. 22, 1994)

### SUMMARY OF INVENTION

### Technical Problem

However, since the configuration disclosed in Patent Literature 1 is a complicated configuration in which two filmy bodies are combined and one of the filmy bodies is formed so as to have a concave shape, this configuration cannot be produced easily. Moreover, in the configuration disclosed in Patent Literature 2, since the reinforcing member has a size which causes the reinforcing member to cover the bottom of the tubular elastic body completely, there is a possibility that a corner on the side face of the reinforcing member may invade a human body when curving movement is produced. Furthermore, the configuration disclosed in Patent Literature 3 cannot be produced easily because this configuration is formed of a plurality of different materials.

The present invention implements a safe device with a simple structure by adopting a new structure for a curving operation at the tip part of medical equipment such as an endoscopic device and solves the problems of the above-described existing single incision laparoscopic cholecystectomy, for example, by making it possible to perform the curving operation automatically by a control device.

### Solution to Problem

An elastic tube according to the present invention is an elastic tube used in medical equipment, the elastic tube including: an elastic tube main body having the shape of a long and narrow hollow circular cylinder; a securing portion provided at one sealed tip of the elastic tube main body, the securing portion to which a medical instrument can be attached; and a fixing portion that can fix an inextensible body having flexibility to the elastic tube main body in the longitudinal direction thereof, in which the elastic tube expands or contracts on the side opposite to the inextensible body as a result of the pressure of gas injected into the elastic tube main body being controlled.

### Advantageous Effects of Invention

According to the present invention, since a structure in which gas (for example, air) is encapsulated in a soft elastic tube and the elastic tube is curved by using the pressure of the gas is adopted, even if the elastic tube makes contact with a medical procedure area (for example, an organ), the elastic tube becomes deformed and absorbs the impact caused by the contact, which prevents the medical procedure area from being damaged and ensures safety. Moreover, since the structure of the elastic tube is simple, the elastic tube can be produced at low cost and can be provided as a disposable item (can be thrown away after one use) without constraint, which makes it possible to maintain the clean state.

Furthermore, the present invention has the advantage that, since the pressure of air, for example, is used as a force curving a tube, even when the air leaks from the elastic tube, the air does not contaminate the medical procedure area. In addition, since an elastic tube which is not expanded is used as a thin tube which is inserted into a medical procedure area (the inside of a body) at the time of medical procedures, it is possible to reduce the diameter of the tip part of medical equipment (for example, an endoscope). Moreover, since automatic operation of the tip part of the medical equipment is possible, there is no need for a surgeon (a camera assistant) for operating the tip part of the medical equipment, which eliminates the possibility that the operation of the above surgeon hinders medical procedures (surgery) which are performed by a surgeon (an operator).

As described above, the present invention offers highly-improved convenience in medical practice in that, for example, it is provided as a disposable item and does not invade a human body while adopting a simple structure which can be produced easily at low cost, and it is expected that the present invention will be widely used in medical practice.

### BRIEF DESCRIPTION OF DRAWINGS

FIG. 1 is a diagram depicting the configuration of medical equipment (an endoscopic device) provided with an elastic tube according to an embodiment of the present invention.

FIG. 2 is a perspective view depicting an endoscope portion (a medical equipment portion) of the endoscopic device (the medical equipment) in FIG. 1.

FIG. 3 is a sectional view of the endoscope portion taken on the line A-A in (a) of FIG. 2.

In FIG. 4, (a) is a sectional view of the elastic tube taken on the line B-B in FIG. 3, (b) to (f) are diagrams depicting modified examples of the elastic tube, and (g) to (i) are diagrams, each depicting another modified example of the elastic tube.

In FIG. 5, (a) to (d) are diagrams, each depicting still another modified example of the elastic tube.

In FIG. 6, (a) is a diagram depicting an example of a bellows structure and (b) is a diagram depicting an example of a mesh structure.

FIG. 7 is a sectional view depicting syringe and piston parts.

In FIG. 8, (a) is a diagram indicating the relationship between the pressure P and the angle of curve θ in the elastic tubes depicted in (a) to (c) of FIG. 4 and (b) is a diagram indicating the relationship between the pressure P and the angle of curve θ in the elastic tubes depicted in (d) to (f) of FIG. 4.

In FIG. 9, (a) and (c) are graphs indicating the relationship between the tube internal pressure of the elastic tube and the cross-sectional area of a hollow part of the elastic tube and (b) and (d) are graphs indicating the relationship between the tube internal pressure and the angle of curve of the tube.

FIG. 10 is a front view depicting how medical procedures (surgery) are performed.

In FIG. 11, (a) to (c) are perspective views depicting the outline of a multi-joint bending portion.

FIG. 12 is a front view depicting how existing medical procedures (surgery) are performed.

### DESCRIPTION OF EMBODIMENTS

### General Outline of Medical Equipment

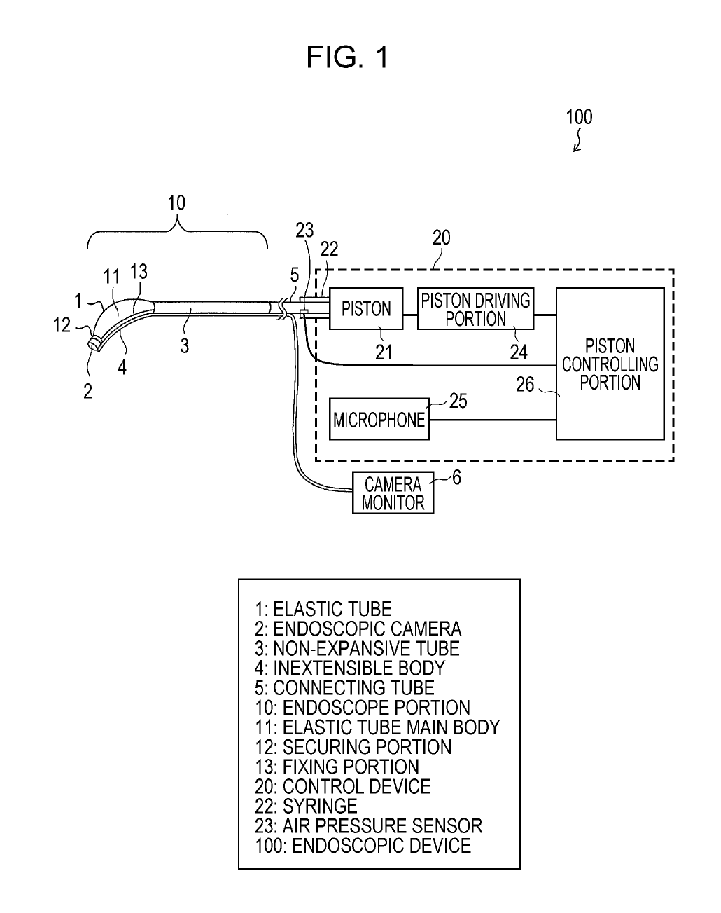

FIG. 1 is a diagram depicting the configuration of an endoscopic device as an example of medical equipment provided with an elastic tube according to an embodiment of the present invention. In FIG. 1, an endoscopic device 100 (medical equipment) includes an endoscope portion (a medical equipment portion) 10 and a control device 20. The control device 20 controls the driving of the endoscope portion 10. The details of the control device 20 will be described later.

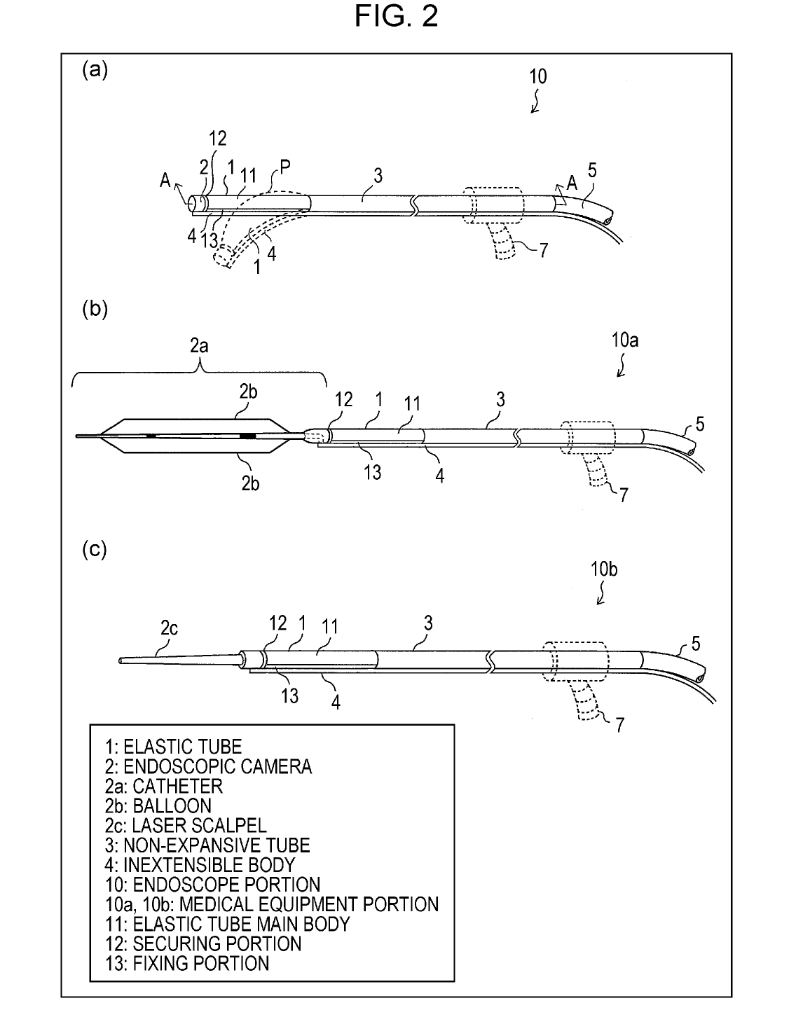

As depicted in FIGS. 2 to 4, the endoscope portion 10 has an expansible elastic tube 1 having the shape of a long and narrow hollow circular cylinder, the elastic tube 1 in which air (gas) W is encapsulated, an endoscopic camera (a medical instrument) 2 secured at the tip of the elastic tube 1, a non-expansive tube 3 that is connected to the other end of the elastic tube 1 in such a way as to communicate therewith and has the shape of a hollow circular cylinder, the non-expansive tube 3 in which the air W (not depicted in the drawing) is encapsulated, and an inextensible body 4 having flexibility, the inextensible body 4 fixed to the elastic tube 1 in the longitudinal direction thereof. Incidentally, the elastic tube 1 in FIG. 1 is in a state in which the elastic tube 1 is expanded by the pressure of the air W which is injected from the control device 20. The expansion and contraction of the elastic tube 1 will be described later in FIGS. 2 and 8.

The elastic tube 1 has an elastic tube main body 11, a securing portion 12, and a fixing portion 13. The elastic tube main body 11 is a main member forming the elastic tube 1 and the tip thereof on the side where the endoscopic camera 2 is secured is sealed. The securing portion 12 is provided at the sealed tip of the elastic tube main body 11 and makes it possible to attach a medical instrument to the elastic tube 1. The medical instrument which is attached to the securing portion 12 is not limited to the endoscopic camera 2 and may be a catheter, a laser scalpel, or the like. The fixing portion 13 is provided on the elastic tube 1 in the longitudinal direction thereof and makes it possible to fix the inextensible body 4 thereto. Incidentally, the securing portion 12 and the fixing portion 13 are part of the elastic tube 1 and formed of the same material as the elastic tube 1.

### (Expansion of the Elastic Tube)

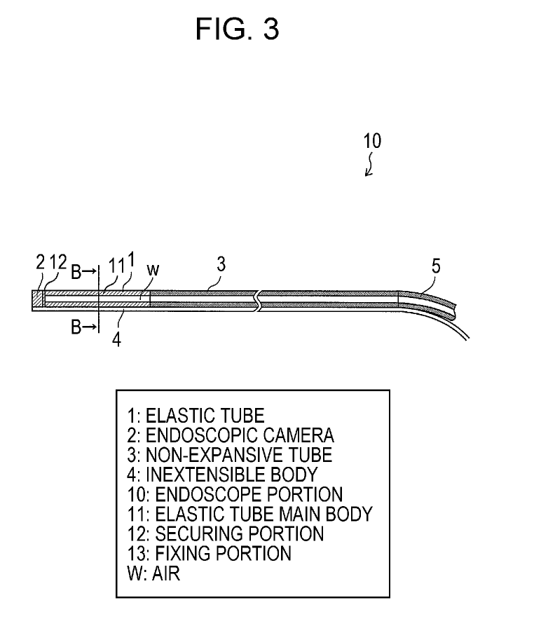

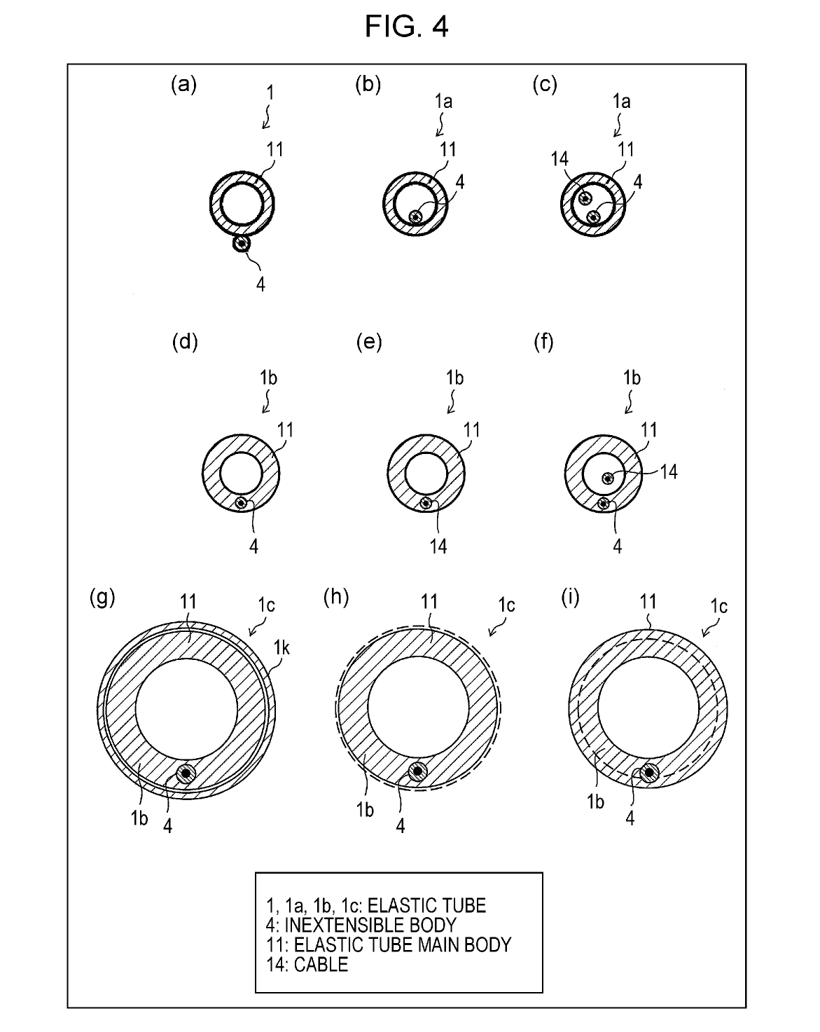

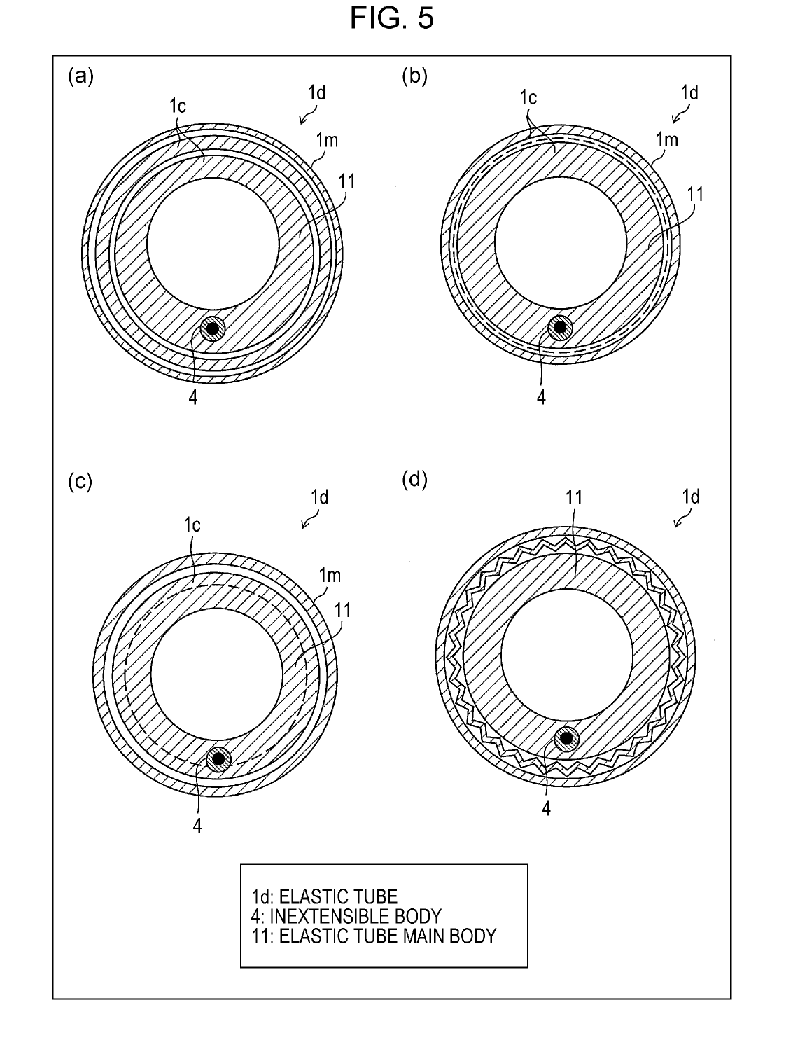

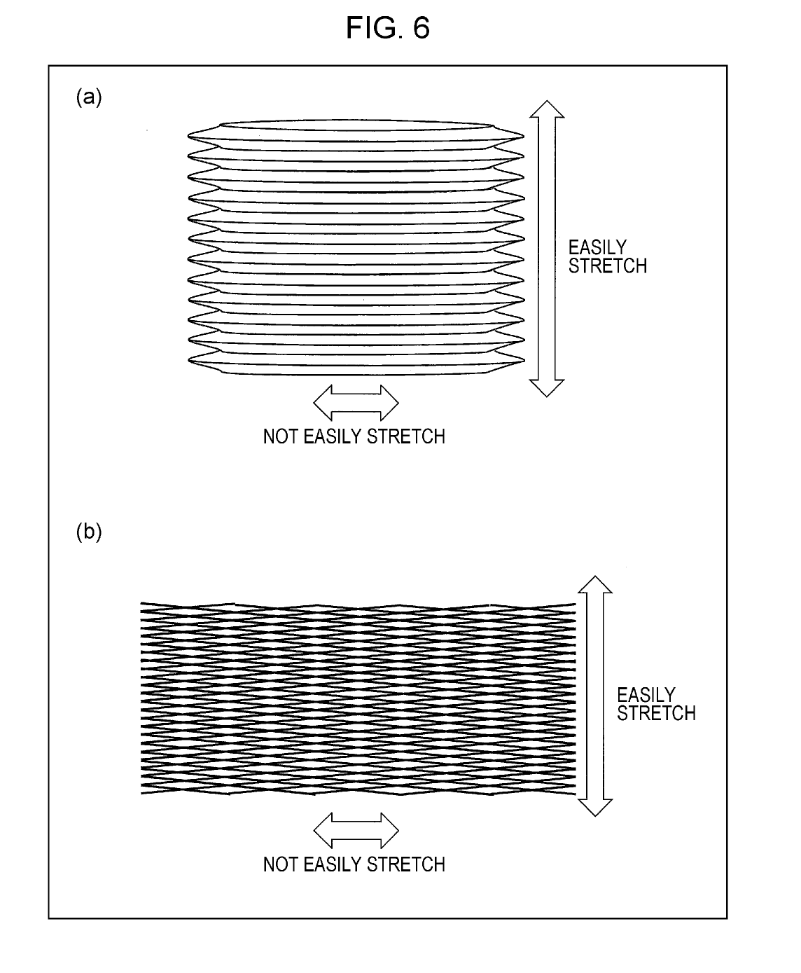

Here, by using FIGS. 2 and 3, the expansion of the elastic tube main body 11 will be described. (a) of FIG. 2 is a perspective view depicting the endoscope portion 10 of the endoscopic device 100 in FIG. 1. FIG. 3 is a sectional view of the endoscope portion 10 taken on the line A-A in (a) of FIG. 2. The elastic tube main body 11 depicted in FIG. 3 is formed as a silicone (polydimethylsiloxane with a toughening agent such as silicon oxide added) tube having an outside diameter of 2 mm and an inside diameter of 1 mm and measuring 5 mm long, for example, and expands and contracts by the pressure of the air W which is injected into the elastic tube main body 11 from the control device 20 (see FIG. 1). When the pressure of the air W encapsulated in the elastic tube main body 11 is normal pressure (1 atmospheric pressure), as depicted by the solid lines in (a) of FIG. 2, the elastic tube 1 has a linear shape. Moreover, by increasing the pressure of the air W encapsulated in the elastic tube main body 11, as depicted by the dashed lines in (a) of FIG. 2, the elastic tube main body 11 expands.

Incidentally, to the securing portion 12 to which the endoscopic camera 2 is attached, a lighting LED lamp (not depicted in the drawing) can be attached in such a way as to be adjacent thereto. Moreover, in the elastic tube 1 depicted in FIG. 3, for the purpose of illustration, the fixing portion 13 is not depicted.

In (a) of FIG. 2, the inextensible body 4 arrests the expansion of the elastic tube main body 11. That is, even when a side of the elastic tube main body 11 opposite to the fixing portion 13 to which the inextensible body 4 is attached and fixed (an upper side of the elastic tube main body 11 above the central line thereof in (a) of FIG. 2) expands (indicated by P in the drawing) as a result of an increase in the pressure of the air W encapsulated in the elastic tube main body 11, expansion does not occur on the side where the fixing portion 13 is located. This allows the elastic tube 1 to curve downward in the drawing (a side opposite to the expanded part P) as depicted by the dashed lines in (a) of FIG. 2. As a result, by changing the pressure of the air W in the elastic tube main body 11, it is possible to change the angle of curve of the elastic tube 1 arbitrarily. Incidentally, the details of the angle of curve of the elastic tube 1 will be described later in FIG. 8.

The extensibility of the inextensible body 4 simply has to be lower than the extensibility of the elastic tube main body 11 and simply has to be high enough to make the elastic tube main body 11 expand on the side opposite to the fixing portion 13 and low enough not to make the elastic tube main body 11 expand on the side where the fixing portion 13 is located. As the material of the inextensible body 4, for example, an inextensible string (a fishing line) such as glass fiber or polyamide fiber may be used or silicone which is the same material as the elastic tube 1 may be used.

Moreover, a cable connected to the medical instrument may double as the inextensible body 4. For example, an electric cord which supplies power to the endoscopic camera 2 may double as the inextensible body 4 depicted in (a) of FIG. 2. Furthermore, a cable connecting the endoscopic camera 2 and the camera monitor 6 along the endoscope portion 10 can double as the inextensible body 4 depicted in FIG. 1. In addition, the inextensible body 4 and the cable connected to the medical instrument can also be fixed to the inside of the elastic tube main body 11. The details will be described later in FIG. 4.

The non-expansive tube 3 is formed as a linear hard hollow body having the same shape as the elastic tube main body 11, that is, having the same outside diameter and inside diameter as the elastic tube main body 11, and measuring about 30 cm long and can be formed of acrylic resin.

In FIG. 1, the connecting tube 5 is a tube connecting the endoscope portion 10 and the control device 20. As depicted in FIG. 2, the connecting tube 5 is a tube having the shape of a hollow circular cylinder and having flexibility and a non-expansive property, the tube connected to the non-expansive tube 3. The connecting tube 5 has almost the same shape as the non-expansive tube 3, measures about 2 m long, and communicates with the elastic tube 1 and the non-expansive tube 3, and, as depicted in FIG. 3, the air W is encapsulated in the connecting tube 5. Incidentally, a flexible stand 7 in (a) to (c) of FIG. 2 is provided on the side of the non-expansive tube 3 where the connecting tube 5 is located. The details of the flexible stand 7 will be described later in FIG. 8.

### (Example of Application of the Medical Instrument)

(b) of FIG. 2 is a perspective view depicting a medical equipment portion 10a as an example of a case where a catheter is attached as the medical instrument. When a catheter 2a is attached to the securing portion 12, a cord which sends air in the catheter 2a can double as the inextensible body 4. As depicted in (b) of FIG. 2, a balloon 2b of the catheter 2a expands by the air sent in the catheter 2a.

Moreover, though not depicted in the drawing, the catheter 2a may be of a guide wire type. In this case, in place of a guide wire for guiding the catheter in a direction in which the catheter should move, the elastic tube 1 is used. That is, to the non-expansive tube 3, the catheter 2a is connected without the elastic tube 1, and, to the other end of the catheter 2a on the side opposite to the non-expansive tube 3, the elastic tube 1 (one end thereof on the side opposite to the securing portion 12) is connected. It is assumed that the gas which is encapsulated in the elastic tube main body 11 is injected from the non-expansive tube 3 via the catheter 2a. As a result, by regulating the pressure of the gas encapsulated in the elastic tube main body 11, the elastic tube 1 can guide the catheter in a direction in which the catheter should move.

(c) of FIG. 2 is a perspective view depicting a medical equipment portion 10b as an example of a case where a laser scalpel is attached as the medical instrument. When a laser scalpel 2c is attached to the securing portion 12, a cord which transmits a signal controlling a laser which is emitted from the laser scalpel 2c can double as the inextensible body 4. Moreover, though not depicted in the drawing, the medical instrument which is attached to the elastic tube 1 may be an electric scalpel.

### (Inside of a Hollow of the Elastic Tube)

As depicted in FIG. 3, the elastic tube 1 and the non-expansive tube 3 are integrated together by bonding and the hollow parts thereof communicate with each other. In the hollow parts, by the control device 20, the air W is encapsulated in the elastic tube 1 via the non-expansive tube 3 from the side where the connecting tube 5 is located.

### Example 1

(a) of FIG. 4 is a sectional view of the elastic tube 1 taken on the line B-B in FIG. 3. This drawing depicts the structure of the elastic tube 1 according to Example 1 of the present invention. The inextensible body 4 is extremely thin as compared to the elastic tube main body 11. For example, if the outside diameter of the cross section of the elastic tube main body 11 is 1 cm, the outside diameter of the cross section of the inextensible body 4 is about 0.5 mm. Incidentally, the cross section of the inextensible body 4 is not limited to a circular cross section and may have a polygonal shape such as a triangular or rectangular shape as long as the corners are chamfered or the size thereof is small so as not to damage a human body when it is inserted into the inside of the body.

Moreover, in (a) of FIG. 4, the elastic tube 1 has a configuration in which the fixing portion 13 (not depicted in the drawing) is placed on the outer periphery (the outside) of the elastic tube main body 11. As a result, the inextensible body 4 is fixed to the outside of the elastic tube main body 11. Incidentally, for the purpose of illustration, in the elastic tube 1 depicted in (a) of FIG. 4, the fixing portion 13 is not depicted.

As depicted in (a) of FIG. 4, if a cable (for example, an electric cord or the like) connected to the medical instrument doubles as the inextensible body 4, the inextensible body 4 (the electric cord) is bonded, in addition to the fixing portion 13 in the elastic tube 1, also to the side face of the non-expansive tube 3 (see FIG. 1) and is led to the outside of the endoscope portion 10.

### Example 2

(b) of FIG. 4 is a diagram depicting a modified example of the elastic tube. This drawing depicts the structure of an elastic tube 1a according to Example 2 of the present invention. The elastic tube 1a has a configuration in which the fixing portion 13 (not depicted in the drawing) is placed on the inner periphery (the inside) of the elastic tube main body 11. As a result, the inextensible body 4 is fixed to the inside of the elastic tube main body 11. Incidentally, the cable connected to the medical instrument may double as the inextensible body 4.

In the above-described configuration, since the inextensible body 4 and the cable connected to the medical instrument are fixed to the inside of the elastic tube main body 11, projections and depressions do not appear on the surface of the elastic tube main body 11. This facilitates the cleaning and disinfection of the elastic tube 1a, which makes it easy to reuse the elastic tube 1a.

Moreover, in the above-described configuration, since the inextensible body 4 and the cable connected to the medical instrument are fixed to the inside of the elastic tube main body 11, the inextensible body 4 and the cable connected to the medical instrument are placed in such a way as to be protected by the elastic tube main body 11. As a result, if part of the inextensible body 4 and the cable connected to the medical instrument is broken for some reasons, the broken part is protected by the elastic tube main body 11, which reduces the possibility of the human body being damaged. For example, if part of the inextensible body 4 is broken for some reasons, even when the extensibility of the inextensible body 4 is lower than the extensibility of the elastic tube 1a, the risk of the human body being damaged by the broken part of the inextensible body 4 is low. Furthermore, for example, if the cable connected to the medical instrument is an electric cable, it is possible to avoid, in the human body, the adverse effects of a leakage of electricity, for example, caused by a breakage of the electric cable. In addition, since gas is injected into the elastic tube main body 11, even when the electric cable is broken, it is possible to avoid, in the elastic tube main body 11, the adverse effects of a leakage of electricity because the inside of the elastic tube main body 11 does not conduct electricity well.

### Example 3

Furthermore, when the inextensible body 4 which is different from the cable connected to the medical instrument is fixed to the elastic tube main body 11 as depicted in (a) and (b) of FIG. 4, the cable connected to the medical instrument may be placed inside a hollow of the elastic tube main body 11. For example, (c) of FIG. 4 is a sectional view of the elastic tube 1a when the fixing portion 13 (not depicted in the drawing) is placed on the inner periphery (the inside) of the elastic tube main body 11. This drawing depicts the structure of the elastic tube 1a according to Example 3 of the present invention. In this case, the inextensible body 4 which is different from the cable connected to the medical instrument is fixed to the inside of the elastic tube main body 11. In addition, a cable 14 connected to the medical instrument is placed inside the hollow of the elastic tube main body 11.

### Examples 4 to 6

(d) to (f) of FIG. 4 are diagrams, each depicting still another modified example of the elastic tube. (d) to (f) of FIG. 4 depict the structures of an elastic tube 1b according to Examples 4 to 6, respectively, of the present invention. The elastic tube 1b may be configured such that the fixing portion 13 is placed inside an elastic body of the elastic tube main body 11. The elastic body forms the elastic tube main body 11 and corresponds to a thickness portion (a diagonally shaded area of FIG. 4) of the elastic tube main body 11 from the inner periphery to the outer periphery thereof. More specifically, the elastic tube 1b has a configuration in which the fixing portion 13 (not depicted in the drawing) is placed between the inner periphery and the outer periphery of the elastic tube main body 11. As a result, as depicted in (d) of FIG. 4, for example, the inextensible body 4 is fixed in a state in which the inextensible body 4 is embedded between the inner periphery and the outer periphery of the elastic tube main body 11. That is, the elastic tube main body 11 depicted in (d) of FIG. 4 can fix the inextensible body 4 to the elastic tube main body 11 as a result of the elastic tube main body 11 being molded in a state in which the inextensible body 4 is integrated with the elastic tube main body 11 in advance. Incidentally, as depicted in (e) of FIG. 4, the cable 14 connected to the medical instrument may double as the inextensible body 4.

Moreover, when the cable connected to the medical instrument does not double as the inextensible body 4, as depicted in (f) of FIG. 4, the inextensible body 4 may be fixed in a state in which the inextensible body 4 is embedded between the inner periphery and the outer periphery of the elastic tube main body 11 and the cable 14 may be placed inside the hollow of the elastic tube main body 11. Incidentally, the inextensible body 4 or the cable 14 may be configured so as to be in contact with the outer periphery and the inner periphery of the elastic tube main body 11. However, in order to enhance the mechanical strength of the elastic tube 1b, as depicted in (d) to (f) of FIG. 4, it is preferable that the inextensible body 4 or the cable 14 is placed between the outer periphery and the inner periphery of the elastic tube main body 11 with a space left between the inextensible body 4 or the cable 14 and the outer periphery and the inner periphery.

In the above-described configuration, since the inextensible body 4 and the cable connected to the medical instrument are embedded between the inner periphery and the outer periphery of the elastic tube main body 11, projections and depressions do not appear on the surface (the outer periphery) of the elastic tube main body 11. This facilitates the cleaning and disinfection of the elastic tube 1b, which makes it easy to reuse the elastic tube 1b.

Furthermore, in the above-described configuration, since the inextensible body 4 and the cable connected to the medical instrument are fixed between the inner periphery and the outer periphery of the elastic tube main body 11 or inside the hollow of the elastic tube main body 11, the inextensible body 4 and the cable connected to the medical instrument are placed in such a way as to be protected by the elastic tube main body 11. As a result, when part of the inextensible body 4 and the cable connected to the medical instrument is broken for some reasons, since the broken part is protected by the elastic tube main body 11, the risk of the human body being damaged is low.

For example, when part of the inextensible body 4 is broken for some reasons, even when the extensibility of the inextensible body 4 is lower than the extensibility of the elastic tube 1b, the risk of the human body being damaged by the broken part of the inextensible body 4 is low. Moreover, for example, when the cable connected to the medical instrument is an electric cable, it is possible to avoid, in the human body, the adverse effects of a leakage of electricity, for example, caused by a breakage of the electric cable. In addition, since gas is injected into the elastic tube main body 11, even when the electric cable is broken, it is possible to avoid, in the elastic tube main body 11, the adverse effects of a leakage of electricity because the inside of the elastic tube main body 11 does not conduct electricity well.

Moreover, in the above-described configuration, the entire outer periphery of the inextensible body 4 or the cable connected to the medical instrument is fixed to the elastic tube main body 11. As a result, the angle of curve θ of the elastic tube 1b changes at a fixed rate in response to the regulation of the pressure P of the air inside the hollow of the elastic tube main body 11. This offers the advantage that the angle of curve θ of the elastic tube main body 11 is easily controlled. The above advantage will be described later in (b) of FIG. 8.

(g) to (i) of FIG. 4 are diagrams, each depicting still another modified example of the elastic tube. (g) to (i) of FIG. 4 depict the structures of an elastic tube 1c according to Examples 7 to 9, respectively, of the present invention. The elastic tube may be formed by combining the elastic tube main body 11 with at least one different elastic tube or a mesh tubular sheet (see a mesh structure depicted in (b) of FIG. 6) which is formed of an elastic body. That is, the elastic tube may have a multiple-layer structure formed of a plurality of elastic tubes or an anisotropic extensible elastic body having, for example, a mesh appearance. Even a single elastic tube can make the extensibility of the elastic tube possess a directional property or the elastic tube possess phased expansion and contraction characteristics by using a multilayered tube, wrapping the mesh tubular sheet around the tube, or embedding the mesh tubular sheet in the tube. As a result, it is possible to control bending movement with high accuracy. In particular, this is highly effective when equal bending is desired to be performed in a plurality of points (for example, when angle 180° bending is performed by performing angle 90° bending in two points or angle 60° bending in three points).

### Example 7

(g) of FIG. 4 depicts the structure of the elastic tube 1c according to Example 7 of the present invention. This drawing is a sectional view of a configuration in which another elastic tube is wrapped around the elastic tube 1b depicted in (d) of FIG. 4. The presence of another elastic tube 1k on the outer perimeter of the elastic tube 1b allows the elastic tube 1c to produce a particular effect of being capable of performing bending effectively by suppressing expansion of the tube in a circumferential direction (a lateral direction) which does not contribute to bending and promoting expansion in a longitudinal direction (a vertical direction) which has a direct influence on the control of the angle of curve. That is, the elastic tube 1c has anisotropic expansion and contraction characteristics with respect to a bending direction. Moreover, since unnecessary expansion of the tube in the circumferential direction is suppressed, degradation of the tube caused by mechanical stress which is produced by repeated expansion and contraction can also be prevented. Incidentally, an outer tube does not necessarily have to have a tube-like shape as long as the outer tube has elasticity. However, in order to enhance ease of cleaning, it is preferable that the outer tube is a tube whose outermost perimeter is a flat surface. Furthermore, it is preferable that the elastic tube has tension possessing a directional property. The circumferential direction of the tube has the function of temporarily suppressing the expansion of an internal tube as a result of having a certain level of tension. It is preferable to adopt a structure or a material which allows the tube to stretch (bend) in the longitudinal direction with a minimum load in response to the expansion of the internal tube.

Furthermore, in the example depicted in (g) of FIG. 4, the number of elastic tubes disposed on the outer perimeter is 1, but the number of elastic tubes is not limited thereto and a plurality of elastic tubes may be disposed on the outer perimeter. In particular, when a plurality of bending points are provided, disposing a plurality of elastic tubes on the outer perimeter is preferable because this makes it possible to perform phased bending movement produced by the application of the tension of the outer tubes and thereby makes it possible to equalize the degrees of bending in the plurality of points.

Here, by using FIG. 9, the behavior of the elastic tube will be described in detail. First, a graph of the relationship between the internal pressure of the tube and the cross-sectional area of the hollow part of the elastic tube in the single elastic tube 1b (see (d) of FIG. 4) serving as the basis is depicted in (a) of FIG. 9 and a graph of the relationship between the internal pressure and the angle of curve in the single elastic tube 1b is depicted in (b) of FIG. 9. As depicted in (a) of FIG. 9, as pressure is applied to the inside of the elastic tube, at first, the elastic tube does not expand in response to the application of pressure due to the tension of the elastic tube. However, the elastic tube starts to expand when the internal pressure exceeds this tension and starts to bend with an increase in the cross-sectional area. This bending movement depends on the thickness of the elastic tube and the modulus of elasticity of the material thereof. For example, as depicted in (a) and (b) of FIG. 9, a soft tube starts to bend from a low pressure and bends greatly; a hard tube starts to bend from a relatively high pressure and bends in a relatively small way. Thus, by setting this structure and the composition of the material arbitrarily, it is possible to set the bending movement characteristics arbitrarily.

Next, (c) of FIG. 9 is a graph of the relationship between the internal pressure and the cross-sectional area of the hollow part of the elastic tube 1b in the elastic tube 1c depicted in (g) of FIG. 4, and (d) of FIG. 9 is a graph indicating the relationship between the internal pressure and the angle of curve of the elastic tube 1b.

As depicted in (c) of FIG. 9, as pressure is applied to the inside of the elastic tube 1b, at first, only a portion corresponding to the elastic tube 1b expands in the elastic tube 1c.

Next, gradual application of pressure is performed, and, when the pressure from the elastic tube 1b is applied to the elastic tube 1k, the expansion is temporarily suppressed.

When application of pressure is further performed, the elastic tube 1b and the elastic tube 1k start to expand at the same time, and full-scale bending starts.

Consequently, the relationship between the pressure and the angle of curve is phased bending movement as depicted in (d) of FIG. 9. That is, the elastic tube 1c exhibits phased expansion and contraction characteristics in response to the magnitude of the internal pressure. Since the above-described bending movement depends not only on the elastic tube 1b, but also on the size and the thickness of the elastic tube 1k and the modulus of elasticity of the material thereof, by arbitrarily setting these structures and the composition of the material, it is possible to set the bending movement characteristics arbitrarily. Incidentally, the above-described phased bending movement of the elastic tube 1c is implemented not only at the time of application (increase) of the internal pressure of the elastic tube 1b, but also at the time of reduction (decrease) of the pressure or at the time of pressure reduction.

Moreover, since the elastic tube 1k is present on the outer perimeter of the elastic tube 1b as described above, it is possible to produce a particular effect of being capable of performing bending effectively by suppressing expansion in the tube circumferential direction (the lateral direction) which does not contribute to bending and promoting expansion in the longitudinal direction (the vertical direction) which has a direct influence on the control of the angle of curve. That is, the elastic tube 1c has anisotropic expansion and contraction characteristics with respect to a bending direction. Moreover, since unnecessary expansion of the tube in the circumferential direction is suppressed, degradation of the tube caused by mechanical stress which is produced by repeated expansion and contraction can also be prevented.

Next, the behavior of this phased bending movement may be adjusted by providing proper clearance between the elastic tube 1k disposed on the outer perimeter and the elastic tube 1b by leaving a space therebetween, or the bending movement may be adjusted by changing the modulus of elasticity of each of the elastic tubes 1b and 1k. In order to change the modulus of elasticity, the material may be changed or an adjustment may be made by changing the thickness. It is preferable to reduce a load on bending by using, for example, a stretchier material or a thinner tube in a position closer to the outer perimeter. Moreover, by adopting a configuration in which the cross section of the outer tube has a corrugated structure and little tension is applied until this corrugated shape becomes flat, an effect equivalent to that obtained by using a hollow tube may be obtained and, by reducing the region of the hollow part, the outside diameter of a bending device may be reduced.

For further enhancement of this effect, the plurality of elastic tubes on the outer perimeter may have a bellows structure (see (a) of FIG. 6) in part thereof. By adopting such a structure, it is possible to further suppress expansion of the tube in the circumferential direction (the lateral direction) which does not contribute to bending and promote expansion in the longitudinal direction (the vertical direction) which has a direct influence on the control of the angle of curve and thereby perform bending more effectively.

Moreover, for the above-described plurality of bending points, the pressure at which bending starts may be regulated by combining the above-described various structures as appropriate such that bending movements in the bending points are performed at the same time or bending is performed in order from a bending point located at the tip, for example.

### Example 8

Next, (h) of FIG. 4 depicts the structure of the elastic tube 1c according to Example 8 of the present invention. This drawing is a sectional view of a configuration in which a mesh tubular elastic sheet (see (b) of FIG. 6), for example, is further wrapped around the outer perimeter of the elastic tube 1b depicted in (d) of FIG. 4. By adopting different mesh crossing angles in the vertical direction and the lateral direction to provide extensibility with a directional property, it is possible to suppress more effectively expansion of the tube in the circumferential direction (the lateral direction) which does not contribute to bending and more effectively promote expansion in the longitudinal direction (the vertical direction) which has a direct influence on the control of the angle of curve and thereby perform bending more effectively. The sheet is not limited to a mesh sheet as long as the sheet provides extensibility with a directional property. Moreover, as in the case of the above description, a plurality of mesh tubular elastic sheets may be wrapped around the outer perimeter and the bending movement may be appropriately adjusted by changing the modulus of elasticity of each mesh tubular elastic sheet.

### Example 9

Next, (i) of FIG. 4 depicts the structure of the elastic tube 1c according to Example 9 of the present invention. This drawing is a sectional view of a configuration in which a mesh tubular elastic sheet is embedded in the elastic tube main body 11. More specifically, the elastic tube 1c has a configuration in which an elastic sheet is placed between the inner periphery and the outer periphery of the elastic tube main body 11. That is, the elastic tube main body 11 depicted in (i) of FIG. 4 can fix the inextensible body 4 and the elastic sheet to the elastic tube main body 11 as a result of the elastic tube main body 11 being molded in a state in which the inextensible body 4 and the elastic sheet are integrated with the elastic tube main body 11 in advance.

Incidentally, in (i) of FIG. 4, a configuration having a single mesh elastic body (elastic sheet) is depicted, but a structure in which a plurality of elastic sheets, for example, two or three stacked elastic sheets, are stacked and embedded may be adopted. For example, by forming the above-described plurality of elastic sheets by using elastic bodies having different expansion and contraction characteristics, it is possible to provide the elastic tube itself with phased expansion and contraction characteristics having a plurality of steps. Since the elastic sheets are integrated together, it is possible to form a compact elastic tube having phased expansion and contraction characteristics and produce the effects of achieving a tube with a smaller diameter and, in addition thereto, achieving a higher degree of expansion and contraction controllability.

Moreover, the cable 14 connected to the medical instrument, the cable 14 depicted in (e) of FIG. 4, may double as the above-described inextensible body 4.

Furthermore, when the cable connected to the medical instrument does not double as the inextensible body 4, as in the drawing depicted in (f) of FIG. 4, the inextensible body 4 may be fixed in a state in which the inextensible body 4 is embedded between the inner periphery and the outer periphery of the elastic tube main body 11 and the cable 14 may be placed inside the hollow of the elastic tube main body 11.

Incidentally, the inextensible body 4 or the cable 14 and the elastic sheet may be configured so as to be in contact with the outer periphery and the inner periphery of the elastic tube main body 11. However, in order to enhance the mechanical strength of the elastic tube 1b, as depicted in (d) to (f) of FIG. 4, it is preferable that the inextensible body 4 or the cable 14 and the elastic sheet are placed between the outer periphery and the inner periphery of the elastic tube main body 11 with a space left between the inextensible body 4 or the cable 14 and the elastic sheet and the outer periphery and the inner periphery.

In the above-described configuration, since the inextensible body 4 or the cable 14 connected to the medical instrument and the elastic sheet are embedded between the inner periphery and the outer periphery of the elastic tube main body 11, projections and depressions do not appear on the surface (the outer periphery) of the elastic tube main body 11. This facilitates the cleaning and disinfection of the elastic tube 1c, which makes it easy to reuse the elastic tube 1c.

Moreover, in the above-described configuration, since the inextensible body 4 and the cable 14 connected to the medical instrument are fixed between the inner periphery and the outer periphery of the elastic tube main body 11 or inside the hollow of the elastic tube main body 11, the inextensible body 4 and the cable 14 connected to the medical instrument are placed in such a way as to be protected by the elastic tube main body 11 and the added elastic sheet. As a result, when part of the inextensible body 4 and the cable 14 connected to the medical instrument is broken for some reasons, since the broken part is protected by the elastic tube main body 11 and the elastic sheet, the risk of the human body being damaged is low.

In the above-described configuration, it is possible to avoid the above-described adverse effects on the human body caused by the cable connected to the medical instrument, for example. Furthermore, since it is possible to make compact an area near the medical instrument secured to the elastic tube 1, the cable connected to the medical instrument does not hinder medical procedures.

Next, (a) to (d) of FIG. 5 are diagrams, each depicting still another modified example of the elastic tube.

### Example 10

(a) of FIG. 5 depicts the structure of an elastic tube 1d according to Example 10 of the present invention. This drawing is a sectional view of a configuration in which another elastic tube 1m is wrapped around the outer perimeter of the elastic tube 1c depicted in (g) of FIG. 4. The presence of the other elastic tube 1m on the outer perimeter of the elastic tube 1c allows the elastic tube 1d to produce a particular effect of being capable of performing bending effectively by suppressing expansion of the tube in the circumferential direction (the lateral direction) which does not contribute to bending and promoting expansion in the longitudinal direction (the vertical direction) which has a direct influence on the control of the angle of curve. Moreover, since unnecessary expansion of the tube in the circumferential direction is suppressed, degradation of the tube caused by mechanical stress which is produced by repeated expansion and contraction can also be prevented.

Next, the behavior of this phased bending movement may be adjusted by providing proper clearance between the elastic tube 1m disposed on the outer perimeter and the elastic tube 1c by leaving a space therebetween, or the bending movement may be adjusted by changing the modulus of elasticity of each of the elastic tubes 1c and 1m. In order to change the modulus of elasticity, the material may be changed or an adjustment may be made by changing the thickness. It is preferable to reduce a load on bending by using, for example, a stretchier material or a thinner tube in a position closer to the outer perimeter. Moreover, by adopting a configuration in which the cross section of the outer tube has a corrugated structure and little tension is applied until this corrugated shape becomes flat, an effect equivalent to that obtained by using a hollow tube may be obtained and, by reducing the region of the hollow part, the outside diameter of the bending device may be reduced.

For further enhancement of this effect, the plurality of elastic tubes on the outer perimeter may have a bellows structure (see (a) of FIG. 6) in part thereof. By adopting such a structure, it is possible to further suppress expansion of the tube in the circumferential direction (the lateral direction) which does not contribute to bending and promote expansion in the longitudinal direction (the vertical direction) which has a direct influence on the control of the angle of curve and thereby perform bending more effectively.

### Example 11

Next, (b) of FIG. 5 depicts the structure of the elastic tube 1d according to Example 11 of the present invention. This drawing is a sectional view of a configuration in which another elastic tube 1m is wrapped around the outer perimeter of the elastic tube 1c depicted in (h) of FIG. 4. The presence of the other elastic tube 1m on the outer perimeter of the elastic tube 1c allows the elastic tube 1d to produce a particular effect of being capable of performing bending effectively by suppressing expansion of the tube in the circumferential direction (the lateral direction) which does not contribute to bending and promoting expansion in the longitudinal direction (the vertical direction) which has a direct influence on the control of the angle of curve. Moreover, since unnecessary expansion of the tube in the circumferential direction is suppressed, degradation of the tube caused by mechanical stress which is produced by repeated expansion and contraction can also be prevented. Furthermore, by appropriately setting the contraction characteristics of the moduli of elasticity of the elastic tube 1m and the mesh elastic sheet, it is possible to perform phased bending movement. In addition, by forming the outermost perimeter as a flat surface, it is possible to enhance ease of cleaning.

### Example 12

Next, (c) of FIG. 5 depicts the structure of the elastic tube 1d according to Example 12 of the present invention. This drawing is a sectional view of a configuration in which another elastic tube 1m is wrapped around the outer perimeter of the elastic tube 1c depicted in (i) of FIG. 4, the elastic tube 1c with the elastic tube main body 11 in which the mesh tubular elastic sheet is embedded. The presence of the other elastic tube 1m on the outer perimeter of the elastic tube 1c allows the elastic tube 1d to produce a particular effect of being capable of performing bending effectively by suppressing expansion of the tube in the circumferential direction (the lateral direction) which does not contribute to bending and promoting expansion in the longitudinal direction (the vertical direction) which has a direct influence on the control of the angle of curve. Moreover, since unnecessary expansion of the tube in the circumferential direction is suppressed, degradation of the tube caused by mechanical stress which is produced by repeated expansion and contraction can also be prevented. Furthermore, by appropriately setting the contraction characteristics of the moduli of elasticity of the elastic tube 1m and the mesh elastic sheet, it is possible to perform phased bending movement.

### Example 13

Next, (d) of FIG. 5 depicts the structure of the elastic tube 1d according to Example 13 of the present invention. This drawing is a sectional view of a configuration in which an outer tube whose cross section has a corrugated structure is wrapped around the outer perimeter of the elastic tube 1b depicted in (d) of FIG. 4 and another outer tube is wrapped around the outer perimeter of the above outer tube. As described above, by adopting a configuration in which the cross section of the outer tube has a corrugated structure and little tension is applied until this corrugated shape becomes flat, an effect equivalent to that obtained by using a hollow tube may be obtained and, by reducing the region of the hollow part, the outside diameter of the bending device may be reduced.

### (Regarding a Multi-Joint Bending Portion)

As depicted in (a) to (c) of FIG. 11, the above-described endoscope portion 10 may be formed as a multi-joint bending portion 30 in which a plurality of bending portions 31 and a plurality of non-bending portions 32 are alternately provided. (a) of FIG. 11 depicts the state of the multi-joint bending portion 30 when the plurality of bending portions 31 are not bent. On the other hand, (b) and (c) of FIG. 11 depict the states of the multi-joint bending portion 30 when each of the plurality of bending portions 31 is bent.

It is desirable to use, as the bending portion 31, an elastic tube provided with a single or a plurality of outer tubes (or elastic sheets) around a single elastic tube main body 11 like the elastic tubes depicted in (g) to (i) of FIG. 4 and (a) to (d) of FIG. 5 described above. Doing so is preferable because this makes it possible to perform phased bending movement which is produced by the application of the tension of the outer tube and thereby equalize the degrees of bending in the bending portions 31 in a plurality of points.

Next, the effect of making the multi-joint bending portion 30 perform bending movement in a plurality of points will be described. As depicted in (b) of FIG. 11, by bending the multi-joint bending portion 30 in such a way that organs and the like do not hinder operation, it is possible to make the multi-joint bending portion 30 perform avoidance operation for organs and treatment tools such as forcipes.

Moreover, in addition thereto, as depicted in (c) of FIG. 11, by rotating the shaft of an elastic tube on the other end which is different from one end at which a camera is installed while making the multi-joint bending portion 30 perform avoidance operation, it is possible to observe an affected part easily from a different direction. At this time, since it is possible to move a camera tip portion while bringing the affected part into view at all times, there is no possibility of losing sight of the affected part which has been brought into view. Thus, this produces a particular effect of being capable of reducing the time (the surgery time) one takes to find the affected part again after losing sight thereof.

### (General Outline of the Control Device)

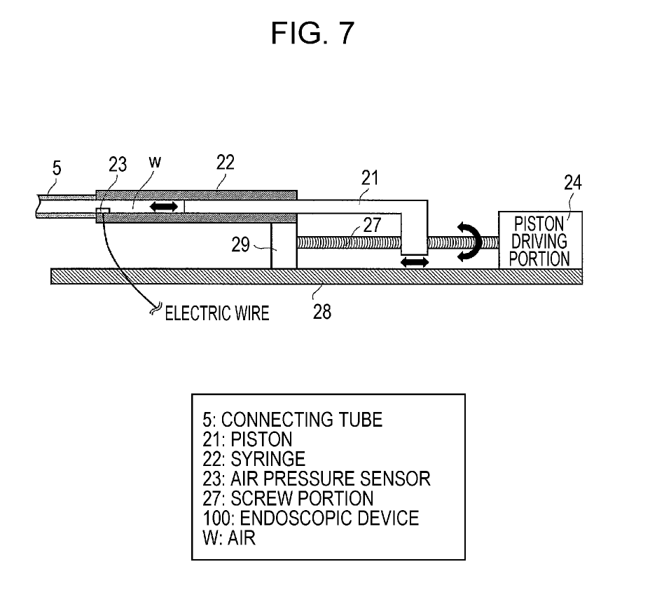

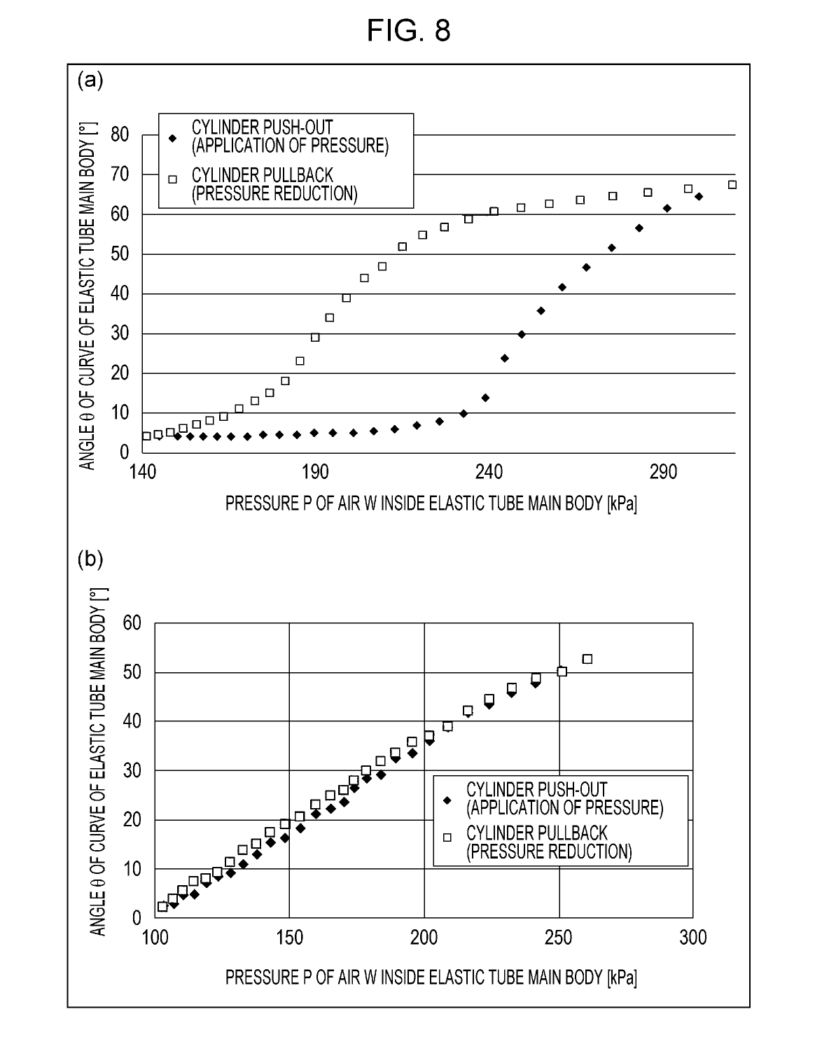

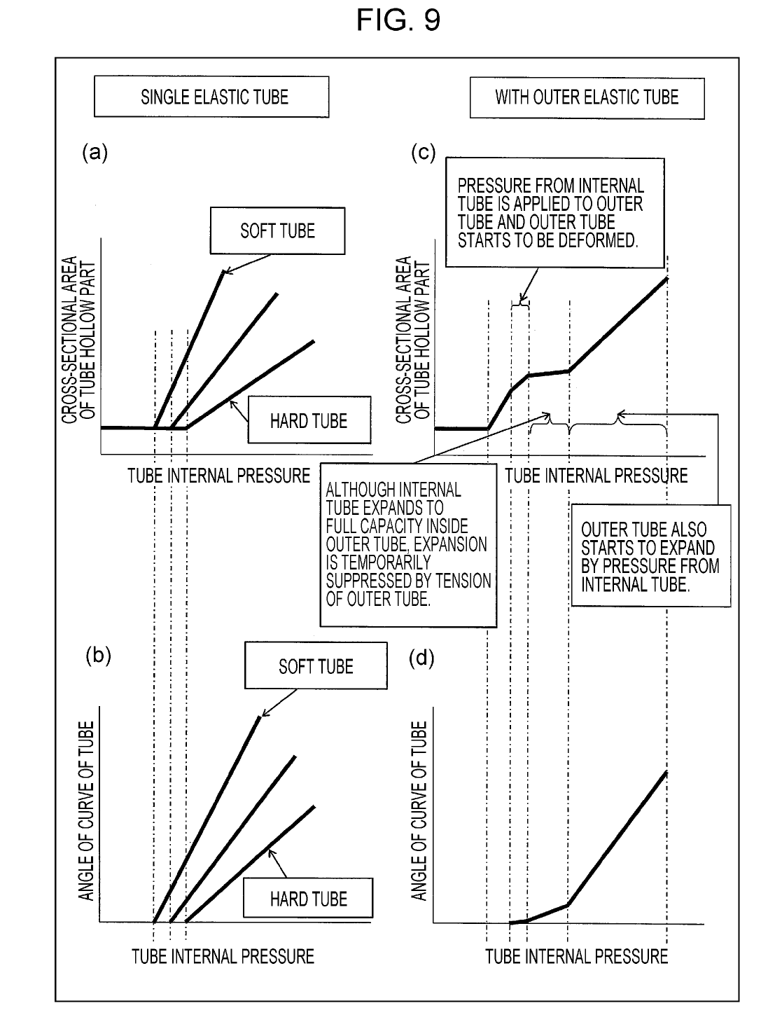

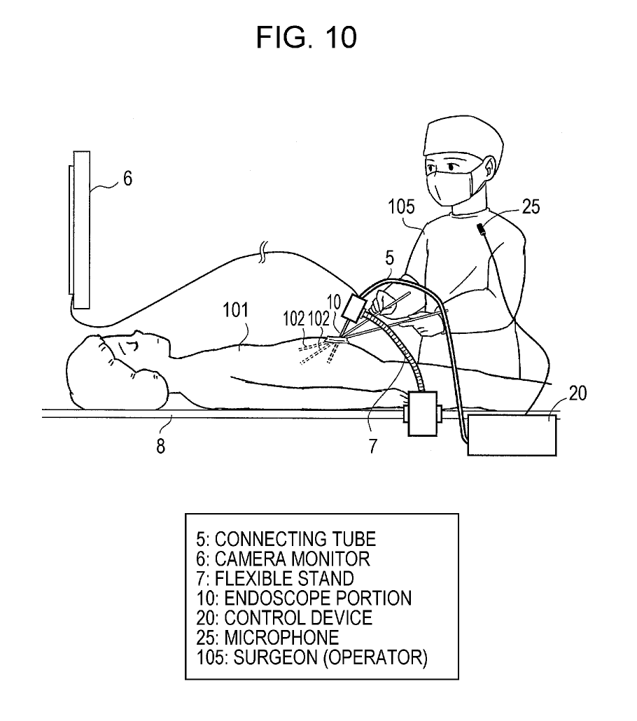

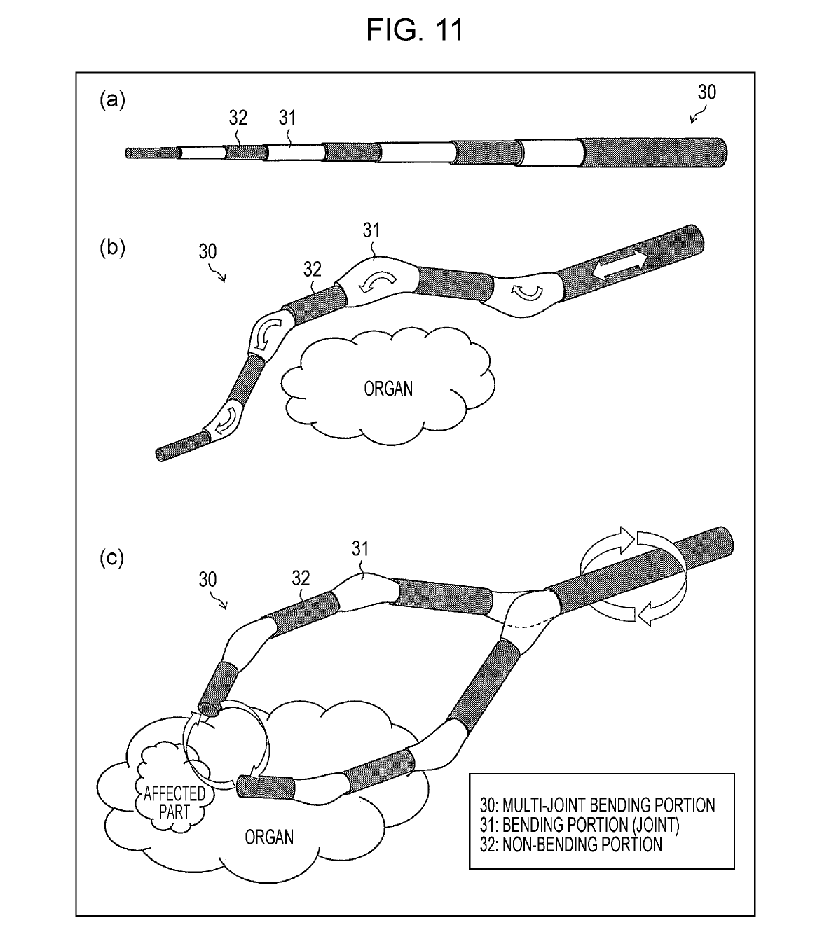

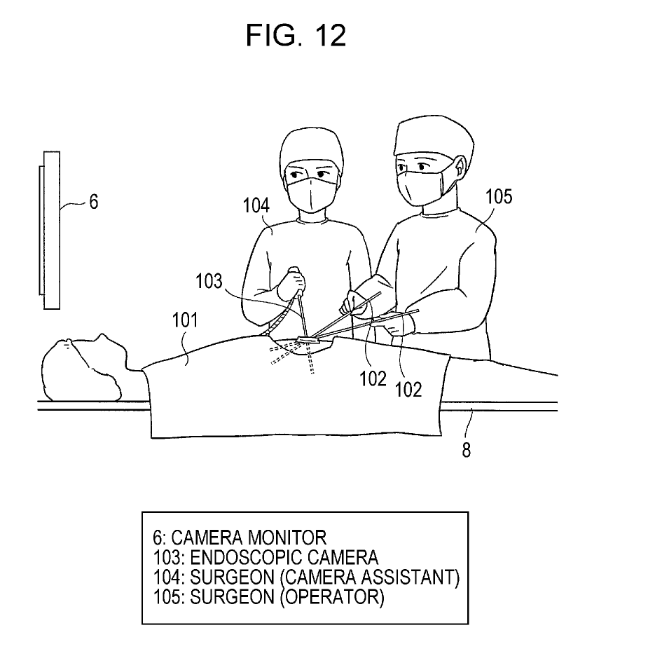

In FIG. 1, the control device 20 will be described. The control device 20 includes a piston (an air pressure variable portion) 21 and a syringe 22 (FIG. 7) which change the pressure of the air (gas) W inside the elastic tube 1, an air pressure sensor 23 which senses the pressure of the air W, a piston driving portion (the air pressure variable portion) 24 which varies the air pressure inside the elastic tube 1 by bringing the piston 21 into operation in the syringe 22, a microphone (an instruction accepting portion) 25 which inputs voice (an instruction) of an operator, and a piston controlling portion (the air pressure variable portion) 26 which controls the piston driving portion 24 by accepting inputs of a voice signal input by the microphone 25 and a detection signal of the air pressure sensor 23. Incidentally, the gas which is encapsulated in the elastic tube 1 is not limited to air, and any gas may be used as long as the gas does not contaminate a medical procedure area.

In this embodiment, as an example of the air pressure variable portion, the piston 21, the piston driving portion 24, and the piston controlling portion 26 are used. However, an air pressure regulating valve (not depicted in the drawing) or the like may be used in place of the piston 21, the piston driving portion 24, and the piston controlling portion 26. Moreover, the microphone 25 is used as an example of the instruction accepting portion, but a foot switch (not depicted in the drawing) or the like may be used in place of the microphone 25.

### (Operation Performed by the Control Device)

FIG. 10 is a front view depicting how medical procedures (surgery) are performed (an example). As depicted in FIG. 10, the piston driving portion 24 is controlled by the piston controlling portion 26 in response to the voice of an operator 105 which is detected by the microphone 25 and the air pressure inside the elastic tube 1 is changed by using the piston 21 and the syringe 22. Incidentally, the details of the piston 21 and the syringe 22 will be described later in FIG. 7.

For example, when the endoscopic camera 2 can curve upward or downward on a display screen of the camera monitor 6, if the operator 105 produces the voice “upward”, the display screen of the camera monitor 6 starts to move so as to display an upper portion of the displayed image. Then, the voice “stop” produced by the operator 105 stops the movement of the display screen on the camera monitor 6. Moreover, if the voice produced by the operator 105 is “downward”, the display screen on the camera monitor 6 moves to a lower portion of the displayed image.

When the display screen of the camera monitor 6 is moved in a horizontal direction, it is necessary to adjust the position of the flexible stand 7 in advance such that the direction in which the elastic tube 1 curves is the horizontal direction. Incidentally, the endoscope portion 10 is maintained in a predetermined position by the flexible stand 7 attached to an operating table 8 and the position is adjusted by the operator 105 by manual operation.

The elastic tube 1 used in the medical procedures and the like depicted in FIG. 10 can be replaced with a new elastic tube 1 (is disposable). Alternatively, the used elastic tube 1 can be used again (the reuse thereof is possible) by cleaning and disinfecting the used elastic tube 1 after the medical procedures are finished. When the elastic tube 1 is reused, in order to prevent the degraded elastic tube 1 from being used, it is necessary to set an upper limit to the number of uses or curves of the elastic tube 1 in the control device 20 in advance and make the elastic tube 1 which exceeds the upper limit unusable.

### (Specific Example of the Control Device)

FIG. 7 depicts the specific structure of the piston 21 and the syringe 22 which perform variable control of the air pressure, and the piston 21 is slidably inserted into the syringe 22 connected to the connecting tube 5. The piston driving portion 24 is a screw portion 27 with one end side (the left side in the drawing) secured to an attachment portion 29 of a base 28 and the other end side (the right side in the drawing) coupled to the piston driving portion 24, and the piston 21 threadably engages the screw portion 27. In the piston driving portion 24, the screw portion 27 is rotated forward or backward, whereby the piston 21 moves in a horizontal direction in the drawing. The forward or backward rotation of the piston driving portion 24 and the number of rotations thereof are controlled by the piston controlling portion 26.

### (Angle of Curve of the Elastic Tube)

FIG. 8 is a diagram indicating the curve characteristics of the elastic tube according to the embodiment of the present invention. The horizontal axis in FIG. 8 represents the pressure (P [kPa]) of the air W inside the elastic tube main body 11, and the vertical axis represents the angle of curve (θ[°]) of the elastic tube main body 11.

(a) of FIG. 8 is a diagram indicating the relationship between the pressure P and the angle of curve θ which is observed when the elastic tubes depicted in (a) to (c) of FIG. 4 are used. As depicted in (a) of FIG. 8, the relationship between the pressure P and the angle of curve θ exhibits hysteresis characteristics (nonlinear characteristics).

For example, when the pressure P of the air W inside the elastic tube main body 11 is increased (at the time of application of pressure), the angle of curve θ of the elastic tube 1 monotonously increases gently in response to an increase in the air pressure to a point near a pressure of 230 kPa. After the pressure P exceeds a point near 230 kPa, the angle of curve θ of the elastic tube 1 sharply increases in response to an increase in the pressure P. Moreover, when the pressure P of the air W inside the elastic tube main body 11 is reduced (at the time of pressure reduction), the angle of curve θ of the elastic tube 1 monotonously decreases gently in response to a reduction in the pressure P to a point near a point at which the pressure P becomes 230 kPa. After the pressure P is reduced to a point near 230 kPa and becomes lower, the angle of curve θ of the elastic tube main body 11 sharply decreases in response to a reduction in the pressure P to a point near a point at which the pressure P becomes 170 kPa. Then, after the pressure P is reduced to a point near 170 kPa and becomes lower, the angle of curve θ of the elastic tube main body 11 monotonously decreases again gently in response to a reduction in the pressure P.

By setting the curve characteristics of the elastic tube main body 11 which are depicted in (a) of FIG. 8 in the piston controlling portion 26 in advance, it is possible to change the angle of curve of the elastic tube main body 11 such that a display image which is displayed on the camera monitor 6 moves at a fixed rate.

(b) of FIG. 8 is a diagram indicating the relationship between the pressure P and the angle of curve θ which is observed when other modified examples of the elastic tube which are depicted in (d) of FIG. 4 to (f) of FIG. 4 are used. As depicted in (b) of FIG. 8, the relationship between the pressure P and the angle of curve θ exhibits nearly linear characteristics.

For example, when the pressure P of the air W inside the elastic tube main body 11 is increased (at the time of application of pressure), the angle of curve θ of the elastic tube 1b increases nearly linearly in response to an increase in the air pressure from a point near a pressure of 100 kPa to a point near 260 kPa. Moreover, when the pressure P of the air W inside the elastic tube main body 11 is reduced (at the time of pressure reduction), the angle of curve θ of the elastic tube 1b decreases linearly in response to a reduction in the pressure P from a point at which the pressure P is 260 kPa to a point near a point at which the pressure P becomes 100 kPa.

As described above, at the time of application of pressure and at the time of pressure reduction, every time the pressure P increases or decreases, the angle of curve θ of the elastic tube 1b changes at each fixed rate, and hysteresis characteristics depicted in (a) of FIG. 8 do not appear. Moreover, the relationship between the pressure P and the angle θ at the time of application of pressure is nearly equal to the relationship between the pressure P and the angle θ at the time of pressure reduction.

By setting the curve characteristics of the elastic tube main body 11 depicted in (b) of FIG. 8 in the piston controlling portion 26 in advance, it is possible to change the angle of curve of the elastic tube main body 11 such that a display image which is displayed on the camera monitor 6 moves at a fixed rate. Furthermore, since the curve characteristics of the elastic tube main body 11 depicted in (b) of FIG. 8 are linear, when the elastic tube main body 11 having linear characteristics is used, the setting of the piston controlling portion 26 becomes simpler than when the elastic tube 1 having hysteresis characteristics depicted in (a) of FIG. 8 is used. This produces the advantage that the angle of curve of the elastic tube main body 11 is easily controlled.

### (Example of Operation of the Elastic Tube)

Moreover, the elastic tube 1 may be configured so as to be operated automatically. For example, when the endoscopic camera 2 depicted in FIG. 1 is used as a medical instrument, a configuration (not depicted in the drawing) in which an image taken by the endoscopic camera 2 is displayed on the camera monitor 6 and, at the same time, the image taken by the endoscopic camera 2 is acquired and analyzed by the control device 20 may be adopted. The control device 20 automatically regulates the pressure of the gas encapsulated in the elastic tube main body 11 in response to the information obtained by analysis. This makes it possible to change the angle of curve of the endoscopic camera 2 automatically. Incidentally, it is assumed that image data and so forth indicating the progress of medical procedures are stored in the control device 20 in advance. In addition, the control device 20 may change the angle of curve of the endoscopic camera 2 by using the hysteresis characteristics depicted in FIG. 8.

Furthermore, the elastic tube 1 may be operated manually through use of a tablet terminal (not depicted in the drawing). For example, when an assistant (not depicted in the drawing) to an operator (for instance, the operator 105 in FIG. 10) who performs medical procedures operates the tablet terminal and changes the angle of curve of the medical equipment, the assistant can operate the tablet terminal in a position away from the operator. As a result, the operation performed by the assistant does not hinder the medical procedure practice performed by the operator, which allows the operator to concentrate on the medical procedures. Moreover, for example, the operator (for example, the operator 105 in FIG. 10) may operate the tablet terminal and change the angle of curve of the medical equipment. In this case, it is possible to make a robot (not depicted in the drawing), in place of the operator, perform actual medical procedures.

### [General Description]

An elastic tube (1) according to a first aspect of the present invention is an elastic tube used in medical equipment (an endoscopic device 100), the elastic tube including: an elastic tube main body (11) having the shape of a long and narrow hollow circular cylinder; a securing portion (12) provided at one sealed tip of the elastic tube main body, the securing portion to which a medical instrument (an endoscopic camera 2, a catheter 2a, a laser scalpel 2c) can be attached; and a fixing portion (13) that can fix an inextensible body (4) having flexibility to the elastic tube main body in the longitudinal direction thereof, in which the elastic tube expands or contracts on the side opposite to the inextensible body as a result of the pressure of gas injected into the elastic tube main body being controlled.

According to the present invention, the above elastic tube offers highly-improved convenience in medical practice in that, for example, it is provided as a disposable item and does not invade a human body while adopting a simple structure which can be produced easily at low cost, and it is expected that the elastic tube will be widely used in medical practice.

In the elastic tube (1) according to a second aspect of the present invention, in the first aspect described above, the fixing portion may fix the inextensible body formed of a material whose extensibility is lower than the extensibility of the elastic tube main body to the elastic tube main body.

In the above-described configuration, the inextensible body formed of a material whose extensibility is lower than the extensibility of the elastic tube main body is fixed to the elastic tube main body. For example, when silicone is used in the inextensible body as the same material as the elastic tube, the inextensible body formed of a silicone material is easily fixed to the elastic tube main body. As a result, even when an unexpected impact caused by contact at the time of medical procedures is given to the elastic tube main body to which the inextensible body formed of a silicone material is fixed, it is possible to prevent the inextensible body formed of the silicone material from being detached from the elastic tube. Moreover, for example, when polyamide fiber is used in the inextensible body, the inextensible body is not easily fixed to the elastic tube formed of a silicone material. This is effective in detaching the inextensible body from the elastic tube with ease.

In the elastic tube (1) according to a third aspect of the present invention, in the first aspect described above, the fixing portion may fix a cable connected to the medical instrument to the elastic tube main body as the inextensible body.

In the above-described configuration, the cable connected to the medical instrument is fixed to the elastic tube main body. This makes it possible to curve the elastic tube by using an object which is used at the time of medical procedures. As a result, the elastic tube has a simple structure in which a new inextensible body is not provided and therefore can be produced easily at low cost.

In the elastic tube (1) according to a fourth aspect of the present invention, in any one of the first to third aspects described above, the fixing portion may be placed between the inner periphery and the outer periphery of the elastic tube main body.

In the above-described configuration, the entire outer periphery of the inextensible body or the cable connected to the medical instrument is fixed to the elastic tube main body. As a result, the angle of curve of the elastic tube changes at a fixed rate in response to the regulation of the pressure P of the air inside a hollow of the elastic tube main body. This offers the advantage that the angle of curve of the elastic tube main body is easily controlled.

In the elastic tube (1) according to a fifth aspect of the present invention, in any one of the first to third aspects described above, the fixing portion may be placed inside a hollow of the elastic tube main body.

In the above-described configuration, since the inextensible body 4 and the cable connected to the medical instrument are fixed to the inside of the elastic tube main body 11, projections and depressions do not appear on the surface of the elastic tube main body 11. This facilitates the cleaning and disinfection of the elastic tube 1a, which makes it easy to reuse the elastic tube 1a.

Moreover, in the above-described configuration, since the inextensible body 4 and the cable connected to the medical instrument are fixed to the inside of the elastic tube main body 11, the inextensible body 4 and the cable connected to the medical instrument are placed in such a way as to be protected by the elastic tube main body 11. As a result, when part of the inextensible body 4 and the cable connected to the medical instrument is broken for some reasons, since the broken part is protected by the elastic tube main body 11, the risk of the human body being damaged is low. For example, if part of the inextensible body 4 is broken for some reasons, even when the extensibility of the inextensible body 4 is lower than the extensibility of the elastic tube 1a, the risk of the human body being damaged by the broken part of the inextensible body 4 is low. Furthermore, for example, if the cable connected to the medical instrument is an electric cable, it is possible to avoid, in the human body, the adverse effects of a leakage of electricity, for example, caused by a breakage of the electric cable. In addition, since gas is injected into the elastic tube main body 11, even when the electric cable is broken, it is possible to avoid, in the elastic tube main body 11, the adverse effects of a leakage of electricity because the inside of the elastic tube main body 11 does not conduct electricity well.

In the elastic tube (1) according to a sixth aspect of the present invention, in the first, second, or fourth aspect described above, the elastic tube main body may place a cable connected to the medical instrument inside a hollow of the elastic tube main body.

In the above-described configuration, even when the inextensible body 4 which is different from the cable connected to the medical instrument is fixed to the elastic tube main body 11 by the fixing portion 13, the cable connected to the medical instrument is placed inside the hollow of the elastic tube main body 11. As a result, it is possible to avoid the above-described adverse effects on the human body caused by the cable connected to the medical instrument. Moreover, since it is possible to make compact an area near the medical instrument secured to the elastic tube 1, the cable connected to the medical instrument does not hinder the medical procedures.

In the elastic tube (1) according to a seventh aspect of the present invention, in any one of the first to sixth aspects described above, the elastic tube may be a tube which is more likely to expand and contract in a longitudinal direction than in a circumferential direction and exhibits different anisotropic expansion and contraction characteristics with respect to a bending direction.

In the above-described configuration, it is possible to produce a particular effect of being capable of performing bending effectively by suppressing expansion of the tube in the circumferential direction (the lateral direction) which does not contribute to bending and promoting expansion of the tube in the longitudinal direction (the vertical direction) which has a direct influence on bending. Moreover, since unnecessary expansion of the tube in the circumferential direction is suppressed, degradation of the tube caused by mechanical stress which is produced by repeated expansion and contraction can also be suppressed.

In the elastic tube (1) according to an eighth aspect of the present invention, in the seventh aspect described above, the elastic tube exhibiting different anisotropic expansion and contraction characteristics with respect to the bending direction may have a structure in which a mesh elastic body is fixed.

In the elastic tube (1) according to a ninth aspect of the present invention, in any one of the first to sixth aspects described above, the elastic tube may be a tube exhibiting phased expansion and contraction characteristics in response to the magnitude of an internal pressure.

In the above-described configuration, it is possible to equalize the degrees of bending in a plurality of points.

In the elastic tube (1) according to a tenth aspect of the present invention, in any one of the seventh to ninth aspects described above, the elastic tube may have a multiple-layer structure formed of a plurality of elastic tubes or an anisotropic extensible elastic body having, for example, a mesh appearance.

In the elastic tube (1) according to an eleventh aspect of the present invention, in the tenth aspect described above, the plurality of elastic tubes may be formed of elastic tubes having different moduli of elasticity or anisotropic extensible elastic bodies having different moduli of elasticity.

A control device (20) according to a twelfth aspect of the present invention may be a control device that controls expansion and contraction of the elastic tube according to the first aspect described above and include an instruction accepting portion (a microphone 25) that accepts an instruction to make the elastic tube expand or contract and an air pressure variable portion (a piston 21, a piston driving portion 24, a piston controlling portion 26) that varies the pressure of gas injected into a hollow of the elastic tube main body based on the instruction accepted by the instruction accepting portion.

In the above-described configuration, since the tip part of the medical equipment is operated by the control device, a surgeon (a camera assistant 104) for operating the tip part of the medical equipment is unnecessary and therefore the operation which is performed by the surgeon (the assistant 104) does not hinder medical procedures (surgery) which are performed by another surgeon (an operator 105). This allows the surgeon (the operator 105) to concentrate on the medical procedures (surgery).

In the control device (20) according to a thirteenth aspect of the present invention, in the twelfth aspect described above, the air pressure variable portion may be automatically operated in accordance with information acquired by the medical instrument.

In the above-described configuration, the elastic tube is automatically curved by automatic operation of the air pressure variable portion. For example, when an endoscopic camera is used as the medical instrument, a configuration in which an image taken by the endoscopic camera is displayed on a camera monitor (6) and, at the same time, the image taken by the endoscopic camera is acquired and analyzed by the control device is adopted. Then, in accordance with the information obtained by the analysis by the control device, the air pressure variable portion is automatically operated and the pressure of the gas encapsulated in the elastic tube main body is automatically regulated, which makes it possible to change the angle of curve of the endoscopic camera automatically. Incidentally, it is assumed that image data and so forth indicating the progress of medical procedures are stored in the control device in advance.

As a result, since the medical instrument is automatically moved even when an operator who performs medical procedures does not give an instruction to move the medical instrument, the operator can concentrate on the medical procedures.

Medical equipment (100) according to a fourteenth aspect of the present invention may include the elastic tube in the first aspect described above and the control device in the twelfth aspect described above.

The medical equipment (100) according to a fifteenth aspect of the present invention may include, in the fourteenth aspect described above, an endoscopic camera (2), a catheter (2a), a laser scalpel (2c), or an electric scalpel as the medical instrument.

The above-described configuration makes it possible to provide medical equipment having both the advantage of the elastic tube according to the present invention and the advantage of the control device according to the present invention.

In particular, medical procedures using the endoscopic camera, the catheter, the laser scalpel, or the electric scalpel tend to be performed frequently. Thus, by providing the endoscopic camera, the catheter, the laser scalpel, or the electric scalpel in the medical equipment of the present invention as the medical instrument, it becomes possible to offer the advantages of the present invention more widely in medical practice.

### (Another Description of the Present Invention)

Incidentally, the present invention can also be described as follows.

That is, the elastic tube according to the present invention may have different anisotropic expansion and contraction characteristics with respect to a bending direction.

Moreover, the elastic tube having different expansion and contraction characteristics with respect to the bending direction may have an anisotropic extensible elastic body having, for example, a mesh appearance, the anisotropic extensible elastic body fixed thereto.

Furthermore, the elastic tube according to the present invention may have phased expansion and contraction characteristics with respect to the internal pressure.

In addition, the elastic tube according to the present invention may be configured to have a multiple-layer structure formed of a plurality of elastic tubes or an anisotropic extensible elastic body having, for example, a mesh appearance. Furthermore, the elastic tube according to the present invention may be configured to have a nesting structure formed of a plurality of elastic tubes or an anisotropic extensible elastic body having, for example, a mesh appearance.

Moreover, the elastic tube of the present invention may be configured such that the plurality of elastic tubes are formed of elastic tubes having different moduli of elasticity or anisotropic extensible elastic bodies having, for example, mesh appearance, the anisotropic extensible elastic bodies having different moduli of elasticity.

Furthermore, the endoscopic device according to the present invention can also be described as an endoscopic device including: an expansible elastic tube having the shape of a long and narrow hollow circular cylinder, the elastic tube in which air is encapsulated; a camera secured to the tip of the elastic tube portion, the tip of the elastic tube portion which is sealed; a non-expansive tube that is connected to the other end of the elastic tube portion in such a way as to communicate therewith and has the shape of a hollow circular cylinder, the non-expansive tube in which air is encapsulated; an inextensible body having flexibility, the inextensible body fixed to the elastic tube portion in a length direction; a connecting tube having the shape of a hollow circular cylinder and having flexibility and a non-expansive property, the connecting tube connected to the non-expansive tube portion; and a controlling portion that performs variable control of the air pressure inside the elastic tube via the connecting tube and the non-expansive tube, the endoscopic device in which the air pressure inside the elastic tube is controlled by the controlling portion and the elastic tube is curved at an arbitrary angle by making the elastic tube expand and contract on the side opposite to the inextensible body by the air pressure.

In addition, the endoscopic device according to the present invention may have, in the endoscopic device having the above-described configuration, a configuration in which the controlling portion includes a piston and a syringe which change the air pressure inside the elastic tube, an air pressure sensor which senses the air pressure, a piston driving portion which varies the air pressure by bringing the piston into operation in the syringe, a microphone which inputs voice of an operator, and a piston controlling portion which controls the piston driving portion by accepting inputs of a voice signal input by the microphone and a detection signal of the air pressure sensor and the controlling portion controls the piston driving portion by the piston controlling portion in response to the voice of the operator and changes the air pressure inside the elastic tube.

Moreover, the endoscopic device according to the present invention may have, in the endoscopic device having the above-described configuration, a configuration in which the inextensible body is formed of polyamide fiber.

Furthermore, in the endoscopic device according to the present invention, in the endoscopic device having the above-described configuration, the inextensible body may be an electric cord which supplies power to the camera.

The present invention is not limited to the embodiments described above and can be changed and modified in various ways within the scope of the claims, and any embodiment that is obtained by appropriately combining the technical means disclosed in different embodiments is also included in the technical scope of the present invention. Furthermore, by combining the technical means disclosed in the embodiments, it is possible to create a new technical feature.

### INDUSTRIAL APPLICABILITY

The present invention can be suitably used in elastic tubes, control devices, and medical equipment. In particular, the present invention can be used in medical equipment provided with an endoscopic camera, a catheter, a laser scalpel, or an electric scalpel in medical practice.

### REFERENCE SIGNS LIST

1 elastic tube
1a elastic tube
1b elastic tube
2 endoscopic camera
2a catheter
2b balloon
2c laser scalpel
3 non-expansive tube
4 inextensible body
5 connecting tube
6 camera monitor
7 flexible stand
8 operating table
10 endoscope portion (medical equipment portion)
10a, 10b medical equipment portion
11 elastic tube main body
12 securing portion
13 fixing portion
14 cable
20 control device
21 piston (air pressure variable portion)
22 syringe
23 air pressure sensor
24 piston driving portion (air pressure variable portion)
25 microphone (instruction accepting portion)
26 piston controlling portion (air pressure variable portion)
27 screw portion
100 endoscopic device (medical equipment)

1-15. (canceled)

16: An elastic tube used in medical equipment, the elastic tube comprising:
an elastic tube main body having a shape of a long and narrow hollow circular cylinder;
a securing portion provided at one sealed tip of the elastic tube main body, the securing portion to which a medical instrument can be attached; and
a fixing portion provided at the elastic tube main body in a longitudinal direction thereof, an inextensible body having flexibility having been fixed to the fixing portion, wherein
the fixing section is provided on an inner periphery of the elastic tube main body,
as a result of a pressure of gas injected into the elastic tube main body being controlled, the elastic tube expands or contracts on a side opposite over a hollow to the fixing section to which the inextensible body has been fixed, the elastic tube main body expanding when the pressure of the gas is increased and contracting when the pressure of the gas is reduced.

17: An elastic tube used in medical equipment, the elastic tube comprising:
an elastic tube main body having a shape of a long and narrow hollow circular cylinder;
a securing portion provided at one sealed tip of the elastic tube main body, the securing portion to which a medical instrument can be attached;
a fixing portion provided at the elastic tube main body in a longitudinal direction thereof, an inextensible body having flexibility having been fixed to the fixing portion; and
a mesh tubular elastic sheet placed between an inner periphery and an outer periphery of the elastic tube main body, the elastic sheet having directionality in expansion and contraction, wherein
the fixing section is placed between the inner periphery and the outer periphery of the elastic tube main body,
as a result of a pressure of gas injected into the elastic tube main body being controlled, the elastic tube expands or contracts on a side opposite over a hollow to the fixing section to which the inextensible body has been fixed, the elastic tube main body expanding when the pressure of the gas is increased and contracting when the pressure of the gas is reduced.

18: The elastic tube according to claim 16, wherein
the fixing portion fixes the inextensible body formed of a material whose extensibility is lower than an extensibility of the elastic tube main body to the elastic tube main body.

19: The elastic tube according to claim 16, wherein
the fixing portion fixes a cable connected to the medical instrument to the elastic tube main body as the inextensible body.

20: The elastic tube according to claim 16, wherein
the elastic tube main body places a cable connected to the medical instrument inside a hollow of the elastic tube main body.

21: The elastic tube according to claim 16, wherein
the elastic tube is a tube which is more likely to expand and contract in a longitudinal direction than in a circumferential direction and exhibits different anisotropic expansion and contraction characteristics with respect to a bending direction.

22: The elastic tube according to claim 21, wherein
the elastic tube exhibiting different anisotropic expansion and contraction characteristics with respect to the bending direction has a structure in which a mesh elastic body is fixed.

23: The elastic tube according to claim 16, wherein
the elastic tube is a tube exhibiting phased expansion and contraction characteristics in response to a magnitude of an internal pressure.

24: The elastic tube according to claim 21, wherein
the elastic tube has a multiple-layer structure formed of a plurality of elastic tubes or an anisotropic extensible elastic body.

25: The elastic tube according to claim 24, wherein
the plurality of elastic tubes are formed of elastic tubes having different moduli of elasticity or anisotropic extensible elastic bodies having different moduli of elasticity.

26: A control device that controls expansion and contraction of the elastic tube according to claim 16, the control device comprising:
an instruction accepting portion that accepts an instruction to make the elastic tube expand or contract; and
an air pressure variable portion that varies a pressure of gas injected into a hollow of the elastic tube main body based on the instruction accepted by the instruction accepting portion.

27: The control device according to claim 26, wherein
the air pressure variable portion is automatically operated in accordance with information acquired by the medical instrument.

28: Medical equipment comprising:
an elastic tube including:
an elastic tube main body having a shape of a long and narrow hollow circular cylinder;
a securing portion provided at one sealed tip of the elastic tube main body, the securing portion to which a medical instrument can be attached; and
a fixing portion provided at the elastic tube main body in a longitudinal direction thereof, an inextensible body having flexibility having been fixed to the fixing portion, wherein
the fixing section is provided on an inner periphery of the elastic tube main body,
as a result of a pressure of gas injected into the elastic tube main body being controlled, the elastic tube expands or contracts on a side opposite over a hollow to the fixing section to which the inextensible body has been fixed, the elastic tube main body expanding when the pressure of the gas is increased and contracting when the pressure of the gas is reduced; and
a control device that controls expansion and contraction of the elastic tube, the control device including:
an instruction accepting portion that accepts an instruction to make the elastic tube expand or contract; and
an air pressure variable portion that varies a pressure of gas injected into a hollow of the elastic tube main body based on the instruction accepted by the instruction accepting portion.

29: The medical equipment according to claim 28, wherein
as the medical instrument, the medical equipment includes an endoscopic camera, a catheter, a laser scalpel, or an electric scalpel.
